(19) United States
(12) Patent Application Publication
(10) Pub. No.: US 2024/0285854 A1
(43) Pub. Date: Aug. 29, 2024

(54) CSF THERAPEUTIC DELIVERY SYSTEM

(71) Applicant: EnClear Therapies, Inc., Newburyport, MA (US)

(72) Inventors: Marcie Glicksman, Salem, MA (US); Rajan Patel, Somerville, MA (US); Gregory Martin, Boston, MA (US); Anthony DePasqua, Newburyport, MA (US)

(21) Appl. No.: 18/590,730

(22) Filed: Feb. 28, 2024

Related U.S. Application Data
(60) Provisional application No. 63/449,019, filed on Feb. 28, 2023.

(51) Int. Cl. A61M 5/168; A61M 1/00

(52) U.S. Cl. CPC A61M 5/16877; A61M 1/772; A61M 5/16804; A61M 2202/0464; A61M 2205/3344; A61M 2205/50; A61M 2210/0693

(57) ABSTRACT

A CSF management method is used with a patient having a body with CSF having a natural flow rate. The method determines determining a target volume for a CSF-containing target compartment of a patient from a volumetric image. The method determines a personalized therapeutic infusion volume of a therapeutic material as a function of the target volume and a total therapeutic infusion volume. A CSF circuit is formed to control flow of CSF in the body. The personalized therapeutic infusion volume is added to the CSF via the CSF circuit at a first time. The therapeutic material is directed, via the CSF, toward the target compartment of the patient. The flow of the CSF in the CSF circuit is adjusted to localize the CSF at the target compartment of the patient.

### PRIORITY & RELATED APPLICATIONS

This patent application claims priority from U.S. provisional patent application No. 63/449,019, filed Feb. 28, 2023, entitled, “CSF System,” and naming Marcie Glicksman, Rajan Patel, and Gregory Martin as inventors, the disclosure of which is incorporated herein, in its entirety, by reference.

This patent application is related to U.S. patent application Ser. No. 17/489,625, filed Sep. 29, 2021, entitled “SUBARACHNOID FLUID MANAGEMENT METHOD AND SYSTEM,” and naming Gianna N. Riccardi, William X. Siopes Jr., Marcie Glicksman, Anthony DePasqua, Kevin Kalish, Joshua G. Vose, and Rajan Patel as inventors, which in turn claims priority to U.S. provisional patent application No. 63/084,996, filed on Sep. 29, 2020, and U.S. provisional patent application No. 63/117,975, filed on Nov. 24, 2020, the disclosures all of which are incorporated herein, in their entireties, by reference.

### FIELD OF THE INVENTION

Illustrative embodiments generally relate to medical devices and methods and, more particularly, illustrative embodiments relate to devices and methods for managing subarachnoid fluid, such as cerebrospinal fluid (“CSF”), and/or drug delivery that may be used to treat neurodegenerative disorders.

### BACKGROUND OF THE INVENTION

When delivering a drug intrathecally, it is difficult to ensure that the delivered dosage reaches the target anatomy (e.g., part of the brain correlating to a specific disease, such as the cortical versus subcortical). It also is difficult to verify the actual dosage delivered to the target anatomy, as well as control, in real time, the concentration of the drug in the fluid surrounding the target anatomy.

### SUMMARY OF VARIOUS EMBODIMENTS

In accordance with one embodiment of the invention, a CSF management method is used with a patient having a body with CSF having a natural flow rate. The method determines a target volume of a CSF-containing target compartment of a patient from a volumetric image. A personalized therapeutic infusion volume of a therapeutic material is determined as a function of the target volume and a total therapeutic infusion volume. A CSF circuit is formed to control flow of CSF in the body. Thee personalized therapeutic infusion volume is added to the CSF via the CSF circuit at a first time. The therapeutic material is directed toward the target compartment of the patient via the CSF. The flow of the CSF in the CSF circuit is adjusted to localize the CSF at the target compartment of the patient.

In various embodiments, adjusting the flow of the CSF comprises oscillating the flow of the CSF. Advantageously, the personalized therapeutic infusion volume may be a fraction of the total therapeutic infusion volume. In other words, each of the personalized therapeutic infusion volumes may be less than the total therapeutic infusion volume.

The volume of the personalized therapeutic infusion volume may be less than or the same as the target volume. The target volume includes a delivery volume. In some embodiments, the target volume may include a peripheral volume. Furthermore, the target volume may be adjusted as a function of compliance of the brain tissue, e.g., based on a sensor measurement.

The method may determine a concentration of the therapeutic material in the CSF (e.g., from a first personalized therapeutic infusion volume), and when the concentration drops below a given threshold, a second personalized therapeutic infusion volume may be added to the CSF via the CSF circuit at a second time subsequent to the first time.

The CSF circuit may form a closed loop with the patient. The CSF circuit may include one or more CSF-containing compartments of the patient anatomy. The CSF-containing compartments include one or more of the lateral ventricles, the lumbar thecal sac, the third ventricle, the fourth ventricle, and the cisterna magna. In various embodiments, the CSF circuit includes a therapeutic delivery pump and a flow control pump. The CSF circuit may include a port into the patient, a pump configured to vary the flow of CSF, and a fluid channel removably coupled with the port and the pump. The fluid channel may be coupled with a flow sensor, a pressure sensor, or both a flow sensor and a pressure sensor.

The therapeutic material may include a drug. The method may mix the therapeutic material and the CSF in a mixing chamber. Localizing may include oscillating the flow of CSF within the CSF circuit for a prescribed time and at a prescribed frequency.

In accordance with yet another embodiment, a CSF management system includes a catheter configured to fluidly couple with a CSF-containing space of a patient and a drug infusion pump. The system also includes a drug infusion pump configured to provide a drug to the patient via the catheter. A controller may be configured to determine a personalized therapeutic infusion volume of therapeutic material to provide to a patient as a function of a received target volume of a CSF-containing target space of the patient. The controller may also be configured to control the drug infusion pump to provide the personalized therapeutic infusion volume to the patient via the catheter. Furthermore, the controller may be configured to adjust flow of the CSF in the patient to localize the CSF at the target space of the patient.

In various embodiments, the pump may be a bidirectional pump. The system may include a target volume calculator configured to determine the target volume from a volumetric image. In various embodiments, the volumetric image may be determined from an MRI scan.

The system may include a therapeutic material position analyzer configured to determine a position of the therapeutic material. The position of the therapeutic material may be used as feedback to control the bidirectional infusion pump to adjust the flow of the CSF to maintain a desired position of the therapeutic material.

The system may also include a drug concentration analyzer configured to determine a concentration of the therapeutic material in the CSF. The drug concentration analyzer may be further configured to use the concentration as feedback to control the delivery of a second personalized therapeutic infusion volume of therapeutic material.

Among other things, the system may include a sensor configured to determine a pressure in the CSF space. The pressure may be used to determine a compliance factor of the brain and adjust the target volume. The bidirectional pump may be configured to adjust the flow by oscillating the flow of the CSF.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a ventricular system of a hypothetical patient in accordance with illustrative embodiments.

FIG. 2 schematically shows a system for targeted and personalized delivery of therapeutics within the CNS in accordance with illustrative embodiments of the invention.

FIG. 3 schematically shows details of the controller of FIG. 2 configured in accordance with illustrative embodiments of the invention.

FIG. 4 shows a process of targeted delivery of a drug using the CSF circuit in accordance with illustrative embodiments of the invention

FIG. 5 schematically shows a two pump circuit with drug fed into pump through separate fluid line in accordance with illustrative embodiments

FIG. 6 schematically shows a two pump circuit with drug introduced directly into fluid line

FIG. 7 schematically shows a flow control valve circuit that may be used with illustrative embodiments.

FIG. 8 schematically shows a syringe pump dosing circuit with a drug introduced directly into the fluid line configured and usable with illustrative embodiments.

FIG. 9 schematically shows a two-pump circuit with a mixing chamber in accordance with illustrative embodiments.

FIG. 10 schematically shows a flow control valve with a mixing chamber in accordance with other embodiments.

FIGS. 11 and 12 schematically show two different user interfaces in accordance with illustrative embodiments.

FIG. 13 schematically shows directing flow from lumbar to ventricle in accordance with illustrative embodiments.

FIG. 14 schematically shows directing flow from ventricle to lumbar in accordance with illustrative embodiments.

FIG. 15 schematically shows directing flow from lumbar to ventricle with a pulsatile pattern in accordance with illustrative embodiments.

FIGS. 16 and 17 schematically show bidirectional pump circuits that enable flow in two opposite directions in accordance with illustrative embodiments.

FIG. 18 schematically shows another system interface in accordance with illustrative embodiments.

FIG. 19 shows a process of manually programming drug delivery in accordance with illustrative embodiments.

FIG. 20 schematically shows an example of a process and an exemplary flow algorithm in accordance with illustrative embodiments.

FIG. 21 shows exemplary data using CSF flow oscillation in the cranium in accordance with illustrative embodiments.

### DESCRIPTION OF ILLUSTRATIVE EMBODIMENTS

In illustrative embodiments, a system controllably applies a therapeutic material, such as a drug (e.g., methotrexate, a chemotherapy and immunosuppressive drug) to a specific anatomical location within the subarachnoid space or other area in a personalized manner. The therapeutic material, which also may be referred to herein as a “drug,” may be applied in one or more doses as a function of the anatomy and physiology of the patient. To that end, the target patient anatomy at which the drug is to be delivered is volumetrically imaged, and a target volume is calculated (e.g., a cranial MRI scan is received and the system determines a volume of one or more ventricles, a portion thereof, the subarachnoid space, etc.). In addition to the volumetric imaging, the targeted volume may also be calculated as a function of the compliance of the brain anatomy.

The system has a controller that manages distribution of the therapeutic material within a CSF circuit through which cerebrospinal fluid (“CSF”) flows in a personalized manner. Specifically, among other things, the controller manages pumps, valves, catheters, and/or other structure(s) to control fluid flow, flow direction, and frequencies of certain periodic flows of bodily fluids (e.g., CSF), to provide a more localized and efficient therapeutic application to a patient as a function of the target volume. The system delivers a total therapeutic infusion volume spread out over multiple personalized doses as a function of the target volume. Advantageously, therapeutic may be delivered in a precise and targeted manner in the patient, while reducing undesirable toxicity and, in some cases, reducing the need for larger volumes of the therapeutic. Details of illustrative embodiments are discussed below.

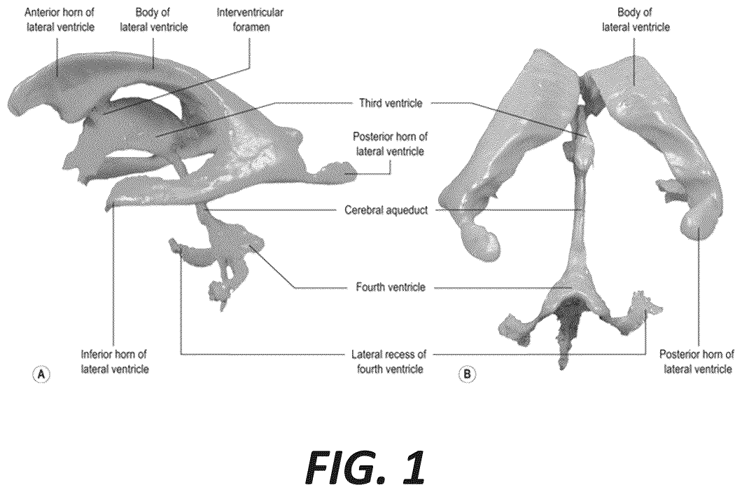

FIG. 1 schematically shows a ventricular system of a hypothetical patient in accordance with illustrative embodiments. The ventricular system forms part of the CFS fluid flow channels within the patient. From top to bottom, the system includes two lateral ventricles, an interventricular foramina, a third ventricle, a cerebral aqueduct, and a fourth ventricle. Cerebrospinal fluid (CSF) flows through the ventricular system of the brain, which consists of four interconnected cavities called ventricles. The path of CSF flow through these ventricles is as follows:
Lateral Ventricles (First and Second Ventricles): CSF is produced by the choroid plexus within the lateral ventricles. From there, it flows through the interventricular foramina (also known as the foramina of Monro) to enter the third ventricle.
Third Ventricle: The CSF then travels through the cerebral aqueduct (also called the aqueduct of Sylvius) into the fourth ventricle.
Fourth Ventricle: From the fourth ventricle, CSF exits into the subarachnoid space via three openings: the paired lateral apertures (foramina of Luschka) and the median aperture (foramen of Magendie).

After leaving the ventricular system, CSF circulates through the subarachnoid space around the brain and spinal cord. It is eventually reabsorbed into the bloodstream through structures called arachnoid villi or granulations, which protrude into the venous sinuses of the brain. This circulation of CSF is important for providing buoyancy and cushioning to the brain and spinal cord, removing waste products, and delivering nutrients to these structures.

Access of therapeutics to the brain is tightly regulated by the blood brain barrier (BBB), severely restricting molecules entering the central nervous system (CNS) via the cerebral circulation. As an alternative drug delivery avenue, therapeutics can be delivered via the Cerebral Spinal Fluid (CSF). There is a fine balance between CSF production (˜20 mL/h), volume (˜150 mL), and removal/turnover (˜every 5 h).

Direct delivery of therapeutics via the CSF through the lateral ventricle or spinal thecal sac is increasingly utilized in clinical practice, but penetration deep into the brain parenchyma is poor and poses a significant barrier. The issue of thigh turnover of CSF volume added to the active efflux to clear molecules makes CNS therapeutics particularly challenging. This has been a significant problem for small molecule and newer CNS therapeutics that do not cross the BBB. Illustrative embodiments advantageously provide a CSF delivery system that may deliver therapeutics broadly within the CNS and may deliver to and penetrate specific CNS targets.

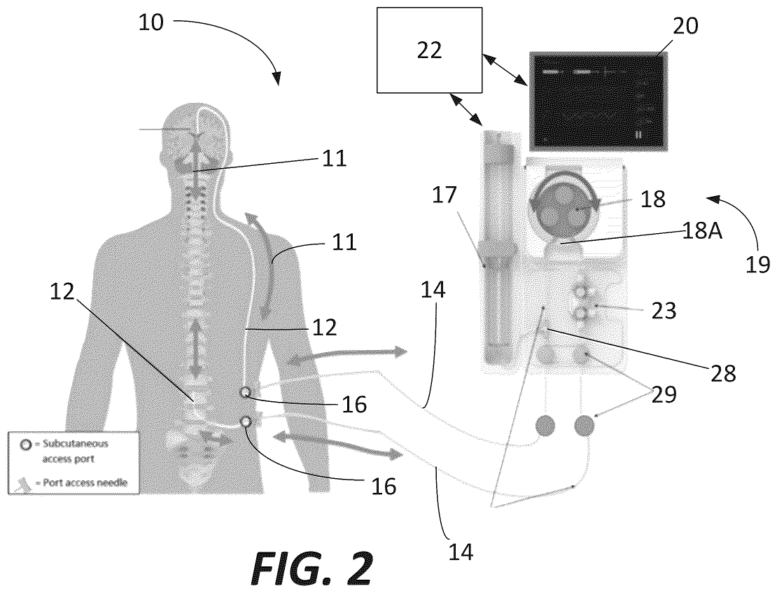

FIG. 2 schematically shows a system 10 for targeted and personalized delivery of therapeutics within the CNS in accordance with illustrative embodiments of the invention. The system 10 may also be referred to as a cerebrospinal fluid circuit 10. As described in greater detail below, CSF flow 11 within the body (e.g., represented by blue arrows through the ventricle) is controlled by the CSF circuit 10.

Internal catheters 12 are positioned in-vivo/interior to the body fluidly couple together via the subarachnoid space. A first internal catheter 12 fluidly couples with a prescribed region of the brain (e.g., the left lateral ventricle via subcutaneous ventricular catheter) to a first port 16, which itself is configured and positioned to be accessible by external components. In a corresponding manner, a second catheter couples the lumbar region or the lower abdomen of the subarachnoid space with a second port 16 that, like the first port 16, also is configured to be positioned and accessible by external components. Accordingly, as will be understood by those skilled in the art, a closed circuit is formed by the system 10 with the patient's body.

The first and second ports 16 may be those conventionally used for such purposes, such as a valved Luer-lock or removable needle. The first and second internal catheters 12 thus may be considered to form a fluid channel extending from the first port 16, to the ventricle, down the spine/subarachnoid space to the lumbar, and then to the second port 16. These internal components, which may be referred to as “internal CSF circuit components,” are typically surgically implanted by skilled professionals in a hospital setting.

The CSF circuit 10 also has external components (referred to as “external CSF circuit components). To that end, the external CSF circuit components may include at least two fluid conduits 14. The external CSF circuit components include a first external fluid conduit 14 that couples with the first port 16 for access to the ventricle. The other end of the first external conduit 14 is coupled with a management system 19, which includes one or more CSF pumps (all pumps are generically identified in the figures as reference number “18”), one or more user interface/displays 20, one or more drug pumps 18, and a control system/controller 22. The external fluid conduit 14 may be implemented as a catheter and thus, that term may be used interchangeably with the term “conduit” and be identified by the same reference number 14.

The management system 19 may be supported by a conventional support structure (e.g., a hospital pole, not shown). To close the CSF circuit 10, a second external catheter 14 extends from that same CSF management system 19 and couples with the second port 16. This management system 19 and the external catheters 14 thus form the exterior part of a closed CSF circuit 10 for circulating the CSF and therapeutic material.

It should be noted that the CSF circuit 10 may have one or more components between the first and second ports 16 and the respective removable connections of the first and second external catheters 14. For example, the first port 16 may have an adapter that couples with the first external catheter 14, or another catheter with a flow sensor may couple between such external catheter 14 and port 16. As such, this still may be considered a removable connection, albeit an indirect fluid connection. There may be corresponding arrangements with the other end of the first external catheter 14, as well as corresponding ends of the second external catheter 14. Accordingly, the connection can be a direct connection or an indirect connection.

The first and second external catheters 14 preferably are configured to removably couple with the management system 19, as well as their respective ports 16. Examples of removable couplings may include a screw-on fit, an interference fit, a snap-fit, or other known removable couplings known in the art. Accordingly, a removable coupling or removable connection does not necessarily require that one forcibly break, cut, or otherwise permanently break the ports 16 for such a connection or disconnection. Some embodiments, however, may enable a disconnection from the first and/or second ports 16 via breaking or otherwise, but the first and/or second ports 16 should remain in-tact to receive another external catheter 14 (e.g., at the end of life of the removed external catheter 14).

The system may include a port configured to receive a drug reservoir 17 (e.g., a single-use syringe) to deliver a dose of therapeutic material (e.g., a drug). The port may fluidly couple with the catheter 14 via a check valve 28 and T-port on the catheter 14. In addition, the catheter 14 is coupled with a bidirectional mechanical pump 18 and also preferably includes a sample port 23 with flow diverters for diverting flow toward or away from a sample port 23. The sample port 23 preferably has sample port flow sensors. Among other things, the system may include two in-line sensor types. The first set of sensors type may be two in-line pressure transducers, manufactured by Utah Medical (DPT-100 Model) one for each access site. A second sensor may be a three-in-one flow, temperature, and bubble detector manufactured by Sensirion (SLFS-300 Model). The three-in-one sensor may be placed closer to the ICV access site. The three-in-one sensor array allows fluid dynamics with extremely high fidelity.

Some embodiments may be implemented as a simple catheter having a body forming a fluid-flow bore with removably couplable ends (or only one removably couplable end). Illustrative embodiments, however, add intelligence to make one or both of these external catheters 14 “smart” catheters, effectively creating a more intelligent flow system. For example, either one or both of the external catheters 14 can have a processor, ASIC, memory, EEPROM (, FPGAs, RFID, NFC, or other logic (generally identified as controller 22) configured to collect, manage, control the device, and store information for the purposes of security, patient monitoring, catheter usage, or communicating with the management system 19 to actively control fluid dynamics of the CSF circuit 10. Among other things, the management system 19 may be configured to coordinate with the controller 22 to control CSF fluid flow via the check valve 28 at the output of the drug reservoir 17. The CSF fluid flow may be controlled as a function of the therapeutic material infusion flow added to the CSF circuit 10, and the target volume, among other things (discussed below).

In addition to the management logic, the external catheter(s) 14 also may have a set of one or more flow sensors and/or a set of one or more pressure sensors. Both of those flow sensors are shown generically at reference number 29, and may be located upstream or downstream from their locations in FIG. 2. For example, the left sensor(s) 29 generically shown in FIG. 2 can be a flow sensor, pressure, or both a flow sensor and pressure. The same can be said for the right sensor(s) 29 generically shown in FIG. 2. They preferably are positioned between the ports 16 on the body and the remaining components as shown. Of course, the flow sensor(s) 29 may be configured to detect flow through the bore of the catheter body, while the pressure sensor(s) 29 may be configured to detect pressure within the bore of the body. Among other functions, the flow sensor(s) 29 may monitor flow rate of fluid through the conduit bore and/or total flow volume through the conduit bore.

The catheter 14 preferably is configured to have different hardness values at different locations. Specifically, illustrative embodiments may use a mechanical pump 18, as shown and noted above. The pump 18 may periodically urge a compressive force along that portion of the catheter 14 it contacts at its interface 18A with the catheter 14. The outlet of the pump 18 in this case may be the portion of the catheter 14 that is receiving the output of a neighboring compressed catheter portion (e.g., a portion that is adjacent to the compressed catheter portion(s). To operate efficiently, illustrative embodiments form the catheter 14 to have a specially configured hardness at that location (e.g., 25-35 Shore A). Diameter also is important for flow and thus, one skilled in the art should determine appropriate diameters as a function of performance and durometer/hardness. Preferably, the catheter portion that contacts the pump 18 is softer than the remainder of the catheter 14, although both could have the same hardness. Accordingly, the catheter preferably has a variable hardness along its length and may even have a variable diameter.

Alternative embodiments may provide an open-loop CSF fluid circuit 10. For example, the CSF fluid circuit 10 may have an open bath (not shown) to which fluid is added and then removed. The inventors expect the closed-loop embodiment to deliver better results, however, than those of the open-loop CSF fluid circuit 10.

Illustrative embodiments are distributed to healthcare facilities and/or hospitals as one or more kits. For example, one more inclusive kit may include the internal and external catheters 12 and 14. Another exemplary kit may include just the internal catheters 12 and the ports 16 (e.g., for a hospital), while a second kit may have the external catheters 14 and/or a single-use syringe. Other exemplary kits may include the external catheters 14 and other components, such as the management system 19 and/or a CSF treatment cartridge 26. See below for various embodiments of the CSF circuit 10 and exterior components that also may be part of this kit.

Accordingly, when coupled, these pumps 18, valves (discussed below and all valves generally identified by reference number 28), internal and external catheters 14, and other components may be considered to form a fluid conduit/channel that directs CSF to the desired locations in the body. It should be noted that although specific locations and CSF containing compartments are discussed, those skilled in the art should recognize that other compartments can be managed (e.g., the lateral ventricles, the lumbar thecal sac, the third ventricle, the fourth ventricle, and/or the cisterna magna). Rather than accessing the ventricle and the lumbar thecal sac, both lateral ventricles could be accessed with the kit. With both internal catheters 12 implanted, CSF may be circulated between the two lateral ventricles, or a drug could be delivered to both ventricles simultaneously.

In illustrative embodiments, the CSF management system 19 generally manages fluid flow to target anatomy through the CSF circuit 10. To that end, that management system 19 has at least one pump 18 (preferably bidirectional, or two unidirectional pumps) that directs flow of the CSF, and at least one pump 18 that directs flow of a therapeutic material (e.g., a drug) though the CSF circuit 10 to desired anatomy. Alternative embodiments may have more pumps 18 for these functions, or combine pumps 18 for these functions. The management system 19 also has a plurality of valves to control flow, and the control system 22, as noted, is configured to control the pumps 18 to selectively apply the drug-carrying CSF to desired local anatomy. FIG. 2 also shows a user interface 20 that enables a clinician to control drug and fluid parameters in the CSF circuit 10 (discussed below) via the control system 22.

Some embodiments may use a monitoring process, such as real-time spectroscopy, to monitor drug concentrations in the CSF. In some of these embodiments, a spectrophotometric sensor may be placed in the CSF circuit 10 to measure the localized concentration of a substance based on its absorption at various wavelengths. For example, some embodiments may use a sensor constructed to measure a single wavelength or multiple wavelengths. The reading taken by the sensor may be relayed to the control system 22, where it is stored or processed. This signal could be processed for a number of purposes, such as to trigger the control system 22 to alter the fluid flow, flow direction, and/or frequencies of certain periodic flows of bodily fluids (e.g., CSF) to provide a more localized and efficient therapeutic application to a patient in real-time. It will be appreciated that the signal could also be stored or displayed such that the changes to flow, direction or frequencies of period flows could be adjust manually.

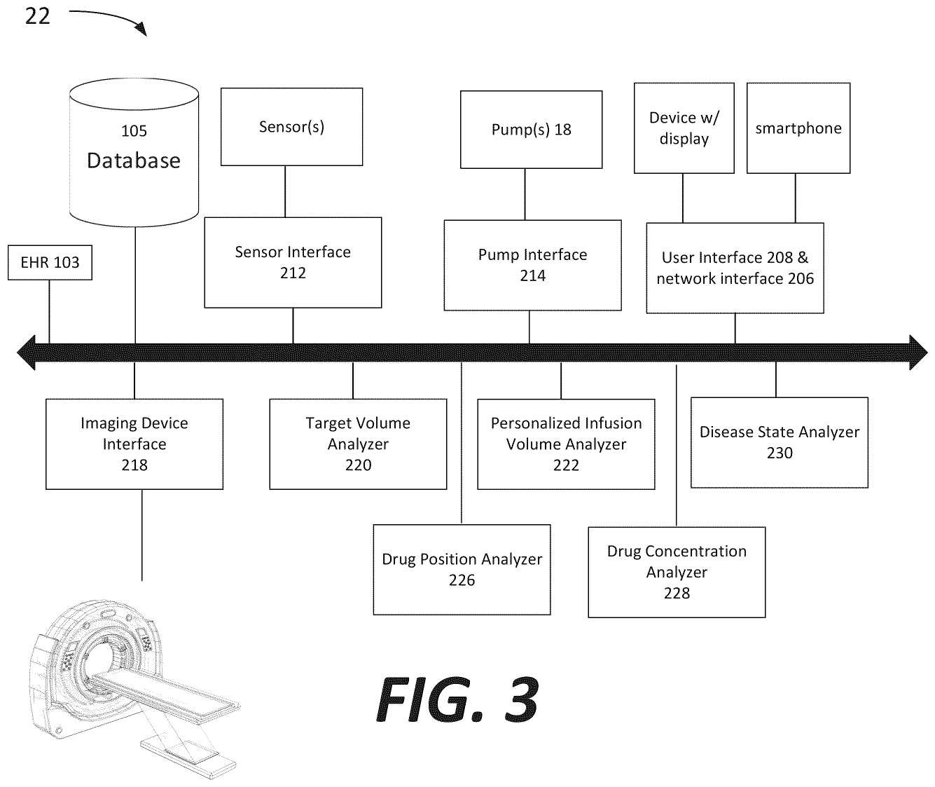

FIG. 3 schematically shows details of the controller 22 of FIG. 2 configured in accordance with illustrative embodiments of the invention. Each of these components is operatively connected by any conventional interconnect mechanism. FIG. 3 simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIG. 3 only schematically shows each of these components. Those skilled in the art should understand that each of these components can be implemented in a variety of conventional manners, such as by using hardware, software, or a combination of hardware and software, across one or more other functional components. For example, the pump interface 214 (discussed in detail below) may be implemented using a plurality of microprocessors executing firmware. As another example, the volume calculator 220 may be implemented using one or more application specific integrated circuits (i.e., “ASICs”) and related software, or a combination of ASICs, discrete electronic components (e.g., integrated circuits), and microprocessors. Accordingly, the representation of the pump interface 214 and other components in a single box of FIG. 3 is for simplicity purposes only. In fact, in some embodiments, the power controller of FIG. 3 is distributed across a plurality of different components—not necessarily within the same housing or chassis.

The controller 22 can be implemented to accomplish a variety of functions. Among others, the controller 22 can ensure that the CSF circuit 10 and its operation is customized/individualized to a patient, a treatment type, a specific disease, and/or a therapeutic material. For example, in response to reading information stored in the database 105, the control system 22 may be configured to control fluid flow as a function of the therapeutic material.

The controller 22 or other logic that manages the external catheter 14 can be configured to provide alerts, and/or produce or cause production of some indicia (e.g., a message, visual indication, audio indication, etc.) indicating that the external catheter 14 has reached an end of its lifecycle, or indicating how much of its lifecycle remains. For example, an external surface of the catheter 14 may have a tag that turns red when the controller 22 and/or other logic determines that the external catheter 14 has reached its full lifetime use. For example, the external catheter 14 may be considered to have a usage meter, implemented as some controller 22, configured to track use of the CSF fluid conduit 14 to help ensure it is not used beyond its rated lifetime. Moreover, the controller 22 may use timers to reduce tampering or use beyond a lifetime.

Some embodiments have a printable circuit board (PCB) equipped with a wireless interface (e.g., Bluetooth antenna) or a hardware connection configured to communicate the pump 18 and/or control system 22. The external catheter 14 can be configured to time out after a certain period, capture data, and communicate back and forth with the control system 22 or other off-catheter or on-catheter apparatus to share system specifications and parameters. The intelligent flow catheter 14 can be designed with proprietary connections such that design of knockoffs or cartridges 26 (discussed below) can be prevented to ensure safety and efficacy of the CSF circuit 10 and accompanying processes.

In various embodiments, the controller 22 may include an imaging device interface 218 configured to communicate with an imaging device. Images or corresponding data from the relevant patient anatomy may be obtained via the imaging device interface 218, from the database 105, and/or from the electronic health record 103.

The images or corresponding data may be accessed by the target volume 220. The target volume analyzer 220 may calculate the target volume, which includes the delivery volume. In some embodiments, the target volume also includes the peripheral volume, and the target volume analyzer 220 also determines the peripheral volume.

In various embodiments, the target volume analyzer 220 accounts for CNS physiology, in particular, compliance of the patient's CNS tissues. receives information relating to anatomical brain compliance. A less compliant brain may require a different flow rate or amount of flow time to have a drug reach the same target than in a more compliant brain, even if overall structures are roughly equal.

To that end, in various embodiments, compliance is measured from the change in pressure (ΔP) following drug volume (ΔV) administration, known also as intracranial elastance (ΔP/ΔV). Compliance is a key element to the understanding patient specific anatomy and physiology, a compliant brain may require an entirely different infusion volume to reach the same target structure, versus a less compliant brain. The patient's disease state is assessed both ahead of time by the patient's neurologist, and conditions such as hydrocephalus can be monitored by the sensor array. The system may report patient CNS compliance from two locations in real time, allowing for a robust calculation of dynamic compliance in real time. This is accomplished by processing the raw pressure waveforms with the sensors. The target volume analyzer 220 may therefore include a compliance factor to adjust the target volume.

A personalized infusion volume analyzer 222 receives the target volume as an input and determines a personalized infusion volume to be provided to the patient. If the target volume is less than the drug volume, the drug volume is divided into multiple sub-doses, each less than or equal to the target volume. Thus, the personalized therapeutic infusion volume is a fraction of the target volume (i.e., TIV=0.9*target volume). The personalized infusion volume analyzer 222 causes the pump 18 to inject the first TIV volume. After that volume has been administered, the pump 18 may be controlled to begin flow oscillation, so the therapeutic is localized at the target volume.

A drug concentration analyzer 228 may communicate with sensors, the database 105, and/or other medical devices (e.g., LC/MS liquid chromatography with the mass analysis capabilities of mass spectrometry in the CSF, spinal cord, and multiple brain regions) to determine a concentration of the infused drug within the CSF fluid pathway or desired target location. When the concentration drops below a certain threshold, a subsequent therapeutic infusion volume may be delivered. This process may be repeated until the total therapeutic volume is delivered.

The personalized infusion volume analyzer 222 communications with the drug concentration analyzer 228. The drug concentration analyzer 228 determines when a concentration of the drug has dropped below a threshold and sends a signal to the personalized volume analyzer 222 to send a subsequent infusion volume. The personalized infusion volume analyzer 222 determines whether there is more drug to be delivered in the total therapeutic volume, and causes the pump to provide an additional therapeutic infusion volume if there is more drug to be delivered.

The controller 22 may include a disease state analyzer 230 that adjusts the personalized infusion volume. The disease state analyzer 230 receives information relating to the patient disease state, if any, and may adjust the therapeutic infusion volume. The disease state information may be obtained from the database 105 or a patient electronic health record 103. For example, a therapeutic for cancer treatment could be for various stages fo disease progression, e.g., active growth, stability, maintenance or therapeutic dose. As such delivery could be modified to suit the clinical scenario.

CSF volumes are determined by disease state, and prior treatments, including brain atrophy caused by disease progression, or chemotherapy and radiation therapy in oncology patients. Or alternatively in patients developing communicating hydrocephalus from their primary disease. Both of these impact intracranial pressure and their cerebral compliance requiring significantly different flow profiles to reach adequate drug delivery and safety thresholds.

The CNS structure and volumetrics are determined by the disease state analyzer 230 in the planning phase of a patient's delivery and are an input into that patient's specific flow algorithm.

A drug position analyzer 226 may communicate with the flow rate sensors, the database 105, and/or other medical devices (e.g., dye imaging device) to determine a position of the infused drug within the CSF fluid pathway. The drug position analyzer 226 may determine that the drug has reached the target location, and begin oscillating fluid flow to maintain the drug in the desired target location.

The controller 22 can be communicate with to one or more pumps 18 configured to provide therapy to the patient (e.g., therapy electrodes 114 as described above) via the pump interface 214. For example, the controller 22 can include, or be operably connected to, circuitry components that are configured to provide infusion. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the controller 22 and under control of one or more processors (e.g., processor) to provide, for example, one or more therapeutic infusions.

In a similar manner, the controller 22 can communicate with one or more sensors configured to monitor one or more physiological parameters of the patient via the sensor interface 212. As shown, the sensors may be coupled to the controller 22 via a wired or wireless connection. The sensors can include one or more electrocardiogram (ECG) electrodes 112 (e.g., similar to sensing electrodes 112 as described above), heart vibrations sensors 224, and tissue fluid monitors 226 (e.g., based on ultra-wide band radiofrequency devices).

All data received from the sensor(s) and/or pump(s) may be stored in the database for subsequent analysis. The data generated may be integrated to form a knowledge database with this novel safety data associated with drug delivery

The database 105 may include data relating to the patient. For example, data that is streamed from the sensors, pumps, or imaging devices can be saved in the database. Additionally, information from any of the other components of the controller can be saved in the database (e.g., target volume, etc.).

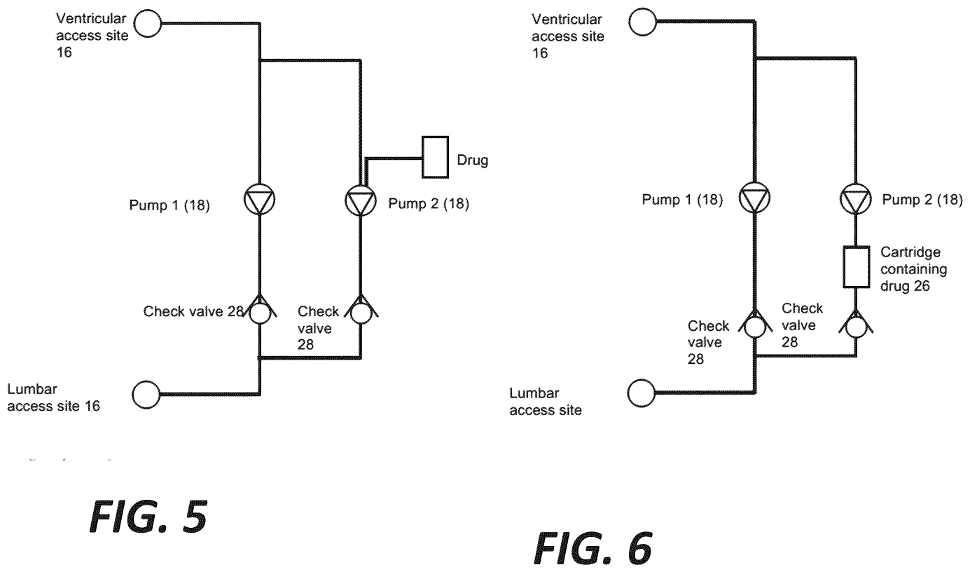

Most patients with CNS disease have images taken of their brain, e.g., MRI scans. These can be stored in the database 105 and used by the controller 22 to generate CSF volumetric analysis, which controls the volumes of therapeutic administered, therapeutic drug delivery infusion rates, CSF flow rates, flow reversal and use of oscillation technique (FIG. 5).

The database 105 can include one or more of non-transitory computer readable media, such as flash memory, solid state memory, magnetic memory, optical memory, cache memory, combinations thereof, and others. The database 105 can be configured to store executable instructions and data used for operation of the controller 22. In certain implementations, the database can include executable instructions that, when executed, are configured to cause the controller 22 to perform one or more functions.

In some examples, the network interface 206 can facilitate the communication of information between the controller 22 and one or more other devices or entities over a communications network. For example, the network interface 206 can be configured to communicate with a remote computing device such as a remote server or other similar computing device. The network interface 206 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the management system 19). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 208 can include one or more physical interface devices such as input devices, output devices, and combination input/output devices and a software stack configured to drive operation of the devices. These user interface elements may render visual, audio, and/or tactile content. Thus, the user interface 208 may receive input or provide output, thereby enabling a user to interact with the controller 22. The user interface may be provided in the form of a graphical user interface.

The GUI may provide a real time moving average, dual y-axis graph of the two pressure sensors raw data in mmHg, and the flow sensor data in mL/min to provide the user with the patient status. The GUI may also provide temperature data and bubble warnings on the display. The capital system and GUI may use audio and visual alarms to alert the user if pressure is outside of safety thresholds (0-20 mmHg) for over 2 minutes.

In some embodiments, the controller 22 can also include at least one battery configured to provide power to one or more components integrated in the controller 22. The battery can include a rechargeable multi-cell battery pack. In one example implementation, the battery can include three or more 2200 mAh lithium ion cells that provide electrical power to the other device components within the controller 22. For example, the battery can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the controller 22.

It should be reiterated that the representation of FIG. 3 is a significantly simplified representation of an actual system controller 22. Those skilled in the art should understand that such a device has other physical and/or functional components, such as central processing units, other packet processing modules, and short-term memory. Accordingly, this discussion is not intended to suggest that FIG. 3 represents all of the elements of the fluid system controller 22. In fact, much of what was said here with regard to FIG. 3 can also be applied to components of the system 10 of FIG. 2.

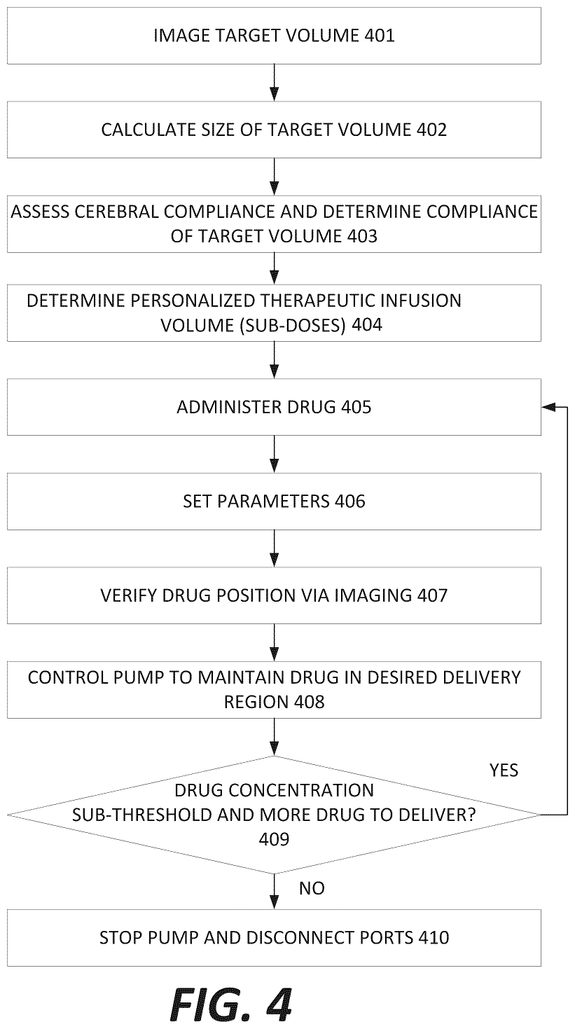

FIG. 4 shows a process of targeted delivery of a drug using the CSF circuit 10 in accordance with illustrative embodiments of the invention. It should be noted that this method is substantially simplified from a longer process that may normally be used. Accordingly, the method shown in FIG. 4 may have many other steps that those skilled in the art likely would use. In addition, some of the steps may be performed in a different order than that shown, or at the same time. Furthermore, in some embodiments, some of these steps are optional. Accordingly, the process is merely exemplary of one process in accordance with illustrative embodiments of the invention. Those skilled in the art therefore can modify the process as appropriate.

The process of FIG. 4 begins at step 401, which volumetrically images the target volume where the drug is to be delivered. A volumetric image refers to one or more images, scans, or data therefrom, that can be used to determine a volume of a CSF containing space (also referred to as a compartment or vessel). For example, it may be desirable to deliver the drug to the third ventricle. The target volume includes the volume of the third ventricle. The process volumetrically images the third ventricle so that the volume of the third ventricle may be determined in subsequent steps. The drug may then be delivered in an appropriate dose as a function of the volumetric imaging of the third ventricle.

In some embodiments, the imaged target volume may include a delivery volume where the drug is to be delivered (e.g., the third ventricle in the above example), as well as a peripheral volume. As explained further below, the peripheral volume includes the volume through which the drug travels as it approaches the delivery volume. The selection of the target volume to include or not include the peripheral volume in the target volume calculation may be made via the user interface 208.

Various embodiments may include a dye or other detectable method to help with appropriate positioning of the drug within the delivery volume. However, some embodiments may not include a dye or other drug positioning assistance. Therefore, in some embodiments, the imaged target volume advantageously includes the volume of CSF between the administration point (e.g., the lateral ventricle for ICV, the lumbar thecal sac for LIT, etc.) and the delivery volume (e.g., the third ventricle). By obtaining the volume between the administration point and the delivery volume (referred to as the peripheral volume), the drug may more accurately be positioned in the appropriate delivery volume. In this example, a larger bolus should be broken up as a function of the volume from the lateral ventricle through the third ventricular volume. However, in some other embodiments, the personalized therapeutic injection volume may be about the volume of the targeted ventricle, or smaller.

In various embodiments, the target volume may be imaged in isolation (e.g., using 3D ultrasound), or as part of a larger image (e.g., cranial MRI scan, full-body CT, etc.). Various embodiments may volumetrically image the target volume using, for example, the following imaging modalities: CT with contrast agent, PET scan, Fluoroscopy using Isoxazol, etc. Illustrative embodiments may operate with any type of volumetric scan/imaging.

The target volume may be volumetrically imaged in parts, e.g., using multiple images or scans (for convenience, various imaging methods are referred to as “scans” without limitation to imaging modality). For example, a scan of the delivery volume (e.g., of the third ventricle) may be obtained, and a separate scan of the peripheral volume (e.g., the volume from the administration point to the lateral ventricle may be obtained). Furthermore, many patients may have scans already taken as part of the course of their normal care.

In various embodiments, data from previous scans can be accessed and used in step 401. As an example, it may be desirable to deliver a drug to the third ventricle of the patient. A cranial MRI scan may be conducted, or a previously conducted cranial MRI scan of the patient may be accessed. These can be used in subsequent steps to generate CSF volumetric analysis, which may determine the volumes of therapeutic administered, therapeutic drug delivery infusion rates, CSF flow rates, flow reversal and CSF directional flow (e.g., oscillation). All of these factors may be optimized to deliver personalized precision treatment to the disease specified target.

At step 402, one or more of these scans, or information therefrom, may be used to calculate the size of the target volume. Volumetric calculations may be obtained from the volumetric imaging from step 401 using known volume calculation engines.

For example, if the target delivery tissue is the basal ganglia, the closest access point is the lateral ventricular catheter. The basal ganglia are located between the lateral ventricles and the third ventricle; therefore, the target would be the third ventricle. The target volume analyzer 220 provides the precise CSF volumes for one lateral ventricle, the interventricular foramen, and the third ventricle (assuming peripheral volume is selected as part of the target volume). Alternatively, the target volume analyzer 220 provides the CSF volumes for the third ventricle.

At step 403, the cerebral compliance of the target volume is determined. The patient's dynamic cerebral compliance may be assessed using the sensor array. Compliance assists with understanding patient specific physiology. For example, a compliant brain may require an entirely different infusion volume to reach the same target structure, versus a less compliant brain. The patient's disease state is assessed ahead of time by the patient's neurology team, and conditions such as hydrocephalus can be monitored by the sensing array.

The system 10 may report patient CNS compliance from two locations in real time, providing a robust calculation of dynamic compliance in real time. Compliance may be determined by processing the raw pressure waveforms using well known methods to determine and report CNS compliance in real time. The sensor array verifies flow rate in real time to ensure accurate delivery.

Pulsatile peak values and increases in nominal pressure with infusion of fluid can be used to calculate a compliance ratio, which may be used by the target volume analyzer 220 to adjust the size of the target volume.

As an example, after compliance is determined, compliance may be used as a factor to adjust the delivery volume. For example, it can be determined based on the patient physiological that the delivery volume is expected to expand by 10% with the increased pressure of the delivered drug. Therefore, the amount of drug delivered may be scaled to 1.1×.

The process then proceeds to step 404, which determines the personalized drug infusion volume. As known by those in the art, a dose of drug can be delivered in a short time period (e.g., 10 seconds, 20 seconds, 60 seconds), or over a longer period (i.e., gradual drug administration). The shorter drug delivery is known in the art as a “bolus” drug delivery.

For example, a prescribed treatment may inject a bolus gene therapy that totals 3,000 microliter. In general, the total drug volume is prescribed by a medical practitioner. The target volume may be determined to be 500 microliter. Illustrative embodiments may divide the total therapy volume of 3,000 microliters into a plurality of sub-doses (i.e., personalized therapeutic infusion volumes) as a function of the target volume (e.g., into 6 separate 500-microliter personalized therapeutic infusion volumes). Advantageously, this avoids injecting all of the drug in a singular space where it otherwise overfills and evacuates the target volume. Of course, other factors may also be considered, and a variety of different infusions could be used. For example, 6 infusions of 500 microliter, 4 infusions of 750 microliter, or 12 infusions of 250 microliter. Preferably, the target volume is set as a maximum volume for drug infusion volume.

In various embodiments, each of the personalized therapeutic infusions volumes is smaller than the anatomical target volume. However, in some embodiments, each of the personalized therapeutic infusion volumes may be larger than the target volume. In some embodiments the personalized therapeutic infusion volume can be 70%, 80%, 90%, or 100% of the target volume. In some other embodiments, the personalized therapeutic infusion volume can be up to 150% of the target volume.

In some embodiments, the total therapeutic infusion volume may be reduced relative to other treatment because the targeted drug delivery process provides high precision delivery of the drug to the targeted tissue (and thus, less waste of the drug to non-target tissues). Alternatively, the total therapeutic infusion volume amount may remain the same. Regardless, the total therapeutic infusion volume may be split up into sub-doses based on the targeted volume.

At step 405, the first therapeutic infusion volume is provided to the patient. The drug may be added to the CSF circuit 10, and/or administered with a tag for imaging to the drug. In the latter example, its position can then be tracked using standard imaging techniques to determine when the drug has reached the target anatomy. Alternative embodiments add the drug to the CSF without administering a tag. Such steps may use other techniques to ensure the drug is localized at the desired target anatomy.

For proper administration, the patient should be fluidly coupled with the CSF circuit 10. In particular, internal catheters 12 are advantageously positioned inside the patient. To access the ventricles and thecal sacs using standard catheters and techniques, thus providing access to the CSF. The access catheters 12 may also be coupled with the peritoneal catheters 12, which are tunneled subcutaneously to the lower abdomen. The tunneled catheters 12 then are connected to the ports 16 implanted in the abdomen.

Additionally, an extracorporeal circulation set (i.e., the external catheters 14, or the “smart catheters” in some embodiments) is coupled with the internal catheters. The extracorporeal circulation set 14 may be primed and connected to the subcutaneous access ports 16. Preferably, this step uses an extracorporeal circulation set, such as one provided by Enclear Therapies, Inc. of Newburyport, MA, and/or the external catheters 14 discussed above. An infusion line or other external catheter 14 is coupled to the management system 19.

At step 406, the parameters are set. In particular the desired flow rate, direction, timing, and other parameters for the CSF circuit 10 to accomplish the application. For example, specific computer program code on a tangible medium within the control system 22 may cooperate with other components of the CSF circuit 10 to control addition of the therapeutic material, localize the therapeutic material, or both.

Step 407 verifies the position of the drug at a target anatomy. When the position of the drug is appropriate, step 408 controls the pump 18 to maintain the drug at that target location. Among other ways, step 408 may control the pump(s) 18 to oscillate at a desired flow rate and frequency to contain the drug at that prescribed or desired anatomical location for a pre-set period of time.

After the therapeutic infusion volume reaches the target anatomy, the pump 18, which can be programmable and/or have logic as discussed above, can reverse CSF flow; specifically, the pump 18 can alternate quickly between pushing and pulling flow of the CSF so that the drug is localized to the target anatomy in the brain (or another target anatomy). In other words, the higher concentration of drug in a portion of the CSF can moved back and forth over the target region. Other embodiments can simply slow down the CSF flow rate to ensure a longer drug application to the target. Either way, these embodiments preferably “soak” the target with the drug, providing a higher quality drug administration. As a result, despite using less of the drug than would be administered by prior art systems, this embodiment still administers a desired amount of the drug to the target by this localizing technique, consequently minimizing toxicity and drug costs.

It should be noted that “reaching” the target anatomy may be defined by the user or other entity within the control system 22. For example, the portion of CSF in the CSF circuit 10 having the higher concentration of drug (from the bolus) may be considered to have reached the target anatomy when some identifiable portion of it (e.g., the highest concentration, or an interior point within the spread of the drug in the CSF) may be within a prescribed distance upstream of the target, or a prescribed distance downstream of the target. Some embodiments may require the defined portion of CSF with the high drug concentration to actually be at or in contact with that target region. Other embodiments may consider the drug to have “reached” the target simply by calculating the time it should take to reach that area, using artificial intelligence/machine learning, and/or through empirical studies.

The process then proceeds to step 409, which asks if the drug-concentration is below a given threshold, and if there is more drug to deliver in the total dosage. If yes, the process returns to step 404, which determines the therapeutic infusion volume for the subsequent infusion. The subsequent infusion may be the same volume as the prior infusion, or may be different. For example, if the total infusion volume is 800 microliter, and the prior infusion provided 500 microliter, then the subsequent infusion can be 300 microliter. In some embodiments, subsequent infusions may deliver reduced therapeutic dosages relative to the prior infusion. Alternatively, subsequent infusions may deliver increased therapeutic dosages relative to the prior infusion. This process is repeated until there is no more drug to deliver.

The process concludes at step 410, which stops the pump 18 when treatment is complete. The management system 19 then may be disconnected and the ports 16 flushed.

In accordance with illustrative embodiments, the CSF circuit 10 is configured to improve the likelihood of the drug passing through the blood-brain barrier. To that end, the management system 19 enables the user or logic to independently set both the flow rate of CSF circulation (e.g., between 0.05 ml/min and 2.0 ml/min, such as 0.5 ml/min) and the dosing rate of the drug (e.g., between 0.01 ml/min to 2.0 ml/min, such as 0.02 ml/min). Preferably, these rates are different, although they can be the same. In illustrative embodiments, the CSF circulation rate is controlled to be different from the natural CSF flow rate. Note that the natural CSF flow rate is the rate of CSF flow without intervention by outside equipment, such as the pumps 18 and other CSF circuit components—even if it is within a range of typical non-interventionally controlled CSF flow rates. Thus, unless the context dictates otherwise, the non-natural CSF flow rate is the flow rate with such intervention. In other embodiments, the CSF flow rate is simply changed from its truly natural flow rate—i.e., the rate at which the CSF flows without intervention.

Depending on a number of factors, the CSF flow rate may be greater than the rate of drug infusion, while in other embodiments, the CSF flow rate is less than rate of the drug infusion rate. Other embodiments may set them to be equal. Those skilled in the art can select the appropriate flow rate based on a variety of factors, including the drug being delivered, the illness, patient profile, rated pressure of the CSF circuit 10, etc.

The inventors recognized that varying the two rates in a coordinated manner enables more control of the drug dose as well as more control of the drug treatment time. Stated another way, these two independent flow rates enable setting of the dosing rate, which allows the user to optimize drug concentration. At the same time, having the ability to set the flow rate allows the user to control the rate of delivery (as opposed to relying upon natural CSF flow).

As noted in the example below, the inventors were surprised to discover that varying the rates in this manner enabled penetration of the drug across the otherwise difficult to penetrate blood-brain barrier. The selected CSF flow rate may be constant or variable. For example, the CSF flow rate may be set to a first rate for a first period of time, a second rate for a second period of time, and a third rate for a third period of time. As such, various embodiments enable flow of the CSF within the CSF circuit 10 at two or more flow rates at two or more different times. The drug delivery rate may be constant or variable in a similar manner, but coordinated with the CSF flow rate to deliver preferred results.

The inventors recognized that a wide variety of different CSF circuit configurations can accomplish the desired goals. FIG. 5 schematically shows a two pump CSF circuit 10 with the drug fed into the pump 18 through a separate fluid line/catheter 12/14 in accordance with illustrative embodiments. In a corresponding manner, FIG. 6 schematically shows a two pump CSF circuit 10 with drug introduced directly into fluid line.

In one embodiment, the CSF circuit 10 has two pumps 18, Pump 1 and Pump 2, to enable a user to set flow rate and dosing rate independently. To that end, Pump 1 may be programmed to control the rate of CSF circulation, while Pump 2 may be programmed to control the dosing rate of the drug to be delivered. Both pumps 18 could be programmed to achieve a desired delivery profile. Check valves 28 or other flow control devices prevent backflow into either pump 18.

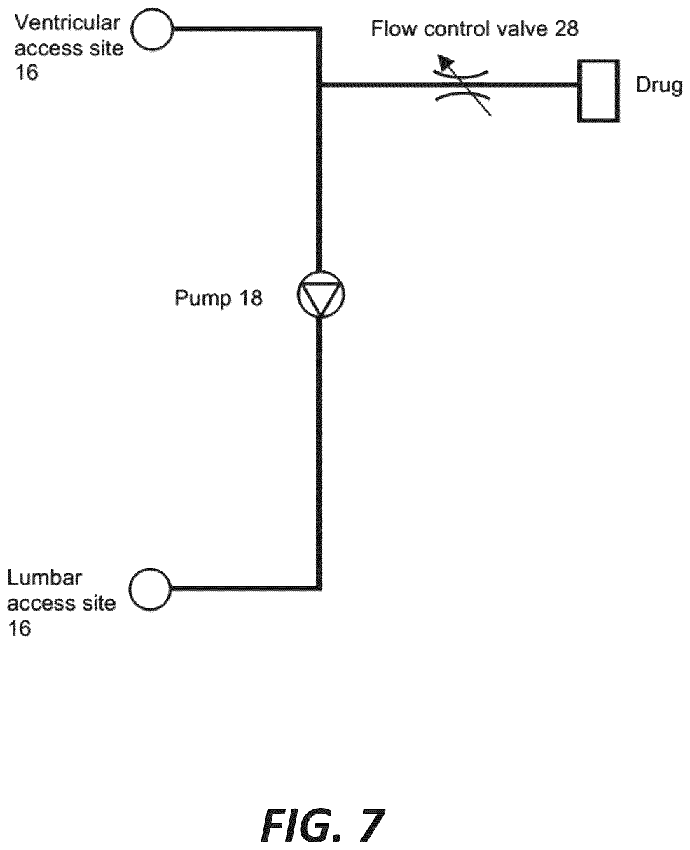

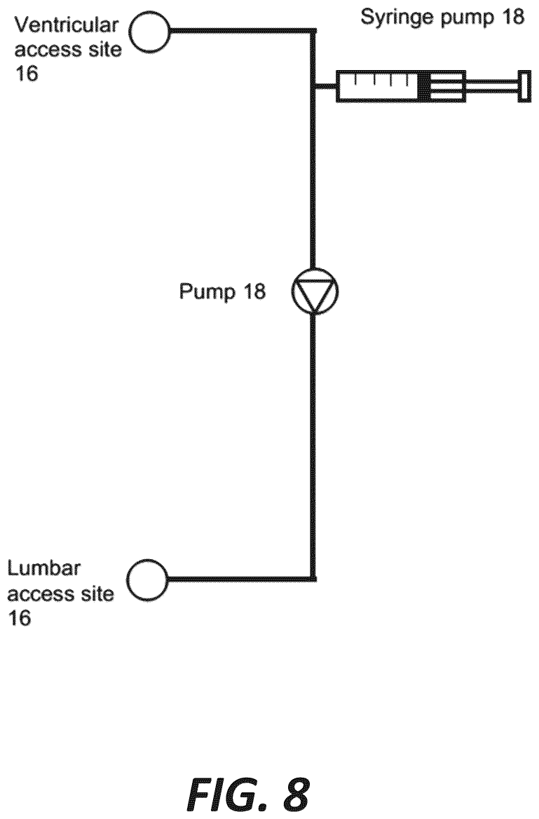

As show, the drug may be fed into the pump 18 through a separate fluid line/catheter (FIG. 5) and input to mix with the patient's CSF in the internal catheter/tubing set 12 before being reintroduced to the body. Alternatively, the drug may also be pre-loaded into a cartridge 26 or other type of drug reservoir and connected directly into the fluid line/catheter 14 (FIG. 6). In this latter embodiment, the CSF mixes with the drug as it flows through tubing set 12/14. FIG. 7 schematically shows another embodiment in which a flow control valve 28 is used in place of Pump 2. In this embodiment, that flow control valve 28 preferably is programmed to control the dosing rate (i.e., the rate of adding the drug to the CSF circuit 10 carrying the CSF.

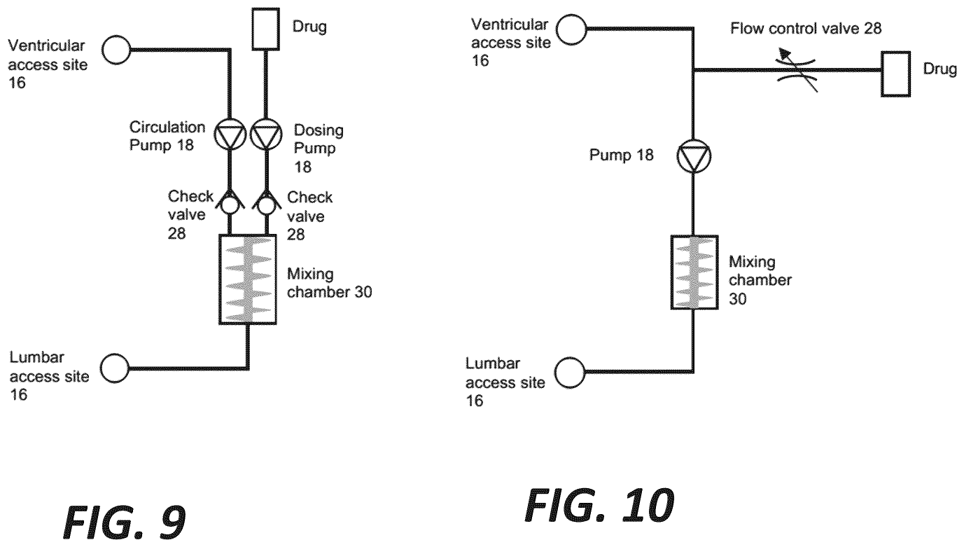

FIG. 9 schematically shows a two-pump circuit 10 with a mixing chamber 30 in accordance with illustrative embodiments. In particular, to ensure a homogeneous mixture of CSF and the drug being delivered, the noted mixing chamber 30 is added to both the two-pump circuit 10 (FIG. 9) and the flow control valve 28 circuit (FIG. 10). The mixing chamber 30 can contain a sensor that provides a readout of a drug's concentration in the CSF, or the management system 19 could simply be programmed to produce a specific drug concentration in the CSF.

In FIG. 9, the CSF-circulating pump 18 and the dosing pump 18 (via an input) feed into the mixing chamber 30 at independent programmable rates. Upon entering the chamber, a small turbine mixes the fluids and the homogeneous mixture is expelled and returned to the patient anatomy. The same concept applies to FIG. 10, but some in-line mixing occurs before the fluids reach the mixing chamber 30.

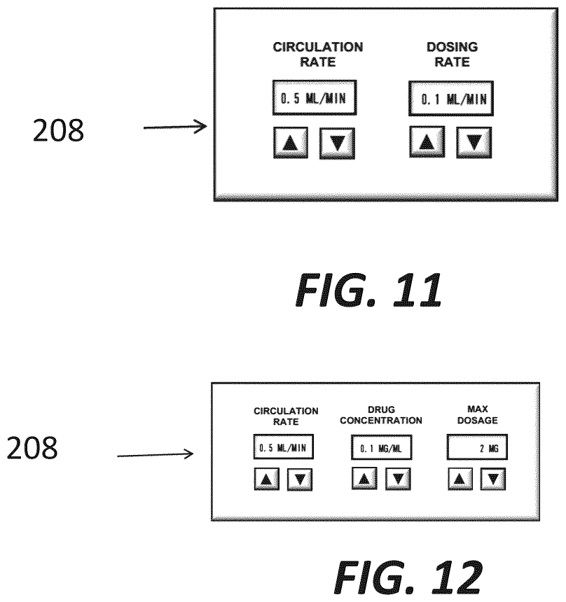

Whether controlling dosing rate by a second pump 18 or by a flow control valve 28, CSF delivery may be manually programmed on an interface/display 20 similar to FIGS. 11 and 12. Specifically, FIGS. 11 and 12 schematically show two different user interfaces 20 in accordance with illustrative embodiments. Rather than requiring the user to input a dosing rate, however, the user may specify a drug concentration and the management system 19 responsively may adjust the dosing rate accordingly to achieve that concentration. The user can also input a maximum dosage. After this dosage was reached, the management system 19 would automatically stop treatment. FIG. 12 shows one such interface 20 (e.g., a graphical user interface or a manual interface).

It should be noted that the during actual processing, the CSF flow rate may differ at different parts of the CSF circuit 10—total CSF flow rate in the CSF circuit 10 is not necessarily homogenous. For example, some parts of the CSF circuit 10 may be wider (e.g., certain human geographies) and thus, may be slower than the average CSF circuit flow rate, while other portions may be narrower, causing a nozzle effect and increasing the CSF flow rate at that point. Near the pump 18 (e.g., at the pump output), however, the CSF flow rate can be controlled to provide a desired rate across the entire CSF circuit 10, even if that rate may deviate in local parts of the CSF circuit.

The discussion above relates to delivering a therapeutic material, such as a drug, over a longer infusion period (e.g., 5 minutes, 10 minutes, 30 minutes, 1-6 hours, days, etc.).

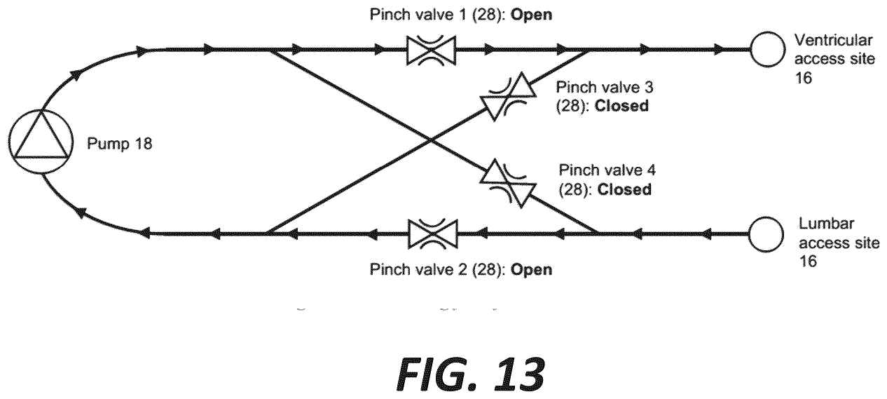

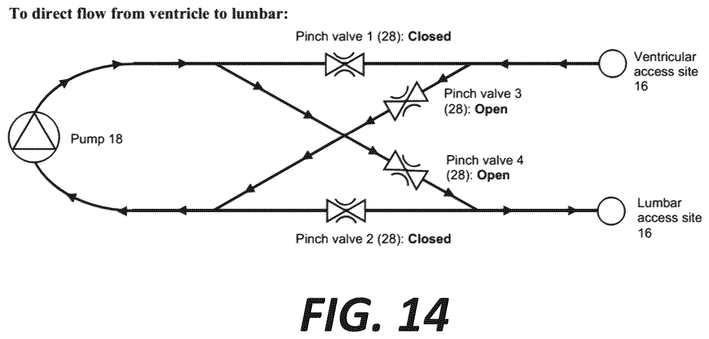

Illustrative embodiments can be implemented in a number of different manners with catheters 12/14, pumps 18, valves 28, etc. similar to those discussed above (including the noted external catheters 14). FIGS. 13-18 show several exemplary implementations. In the embodiment shown in FIG. 13, the CSF circuit 10 has four pinch valves 28 on tubing (i.e., external catheters 14), enabling fluid oscillation between opposing flow directions. To flow from lumbar to ventricle (FIG. 13), pinch valves 1 and 2 are opened while pinch valves 3 and 4 are closed. Conversely, to switch flow direction from ventricle to lumbar (FIG. 14), pinch valves 1 and 2 are closed while pinch valves 3 and 4 are opened. Controlling the pinch valves 28 in this manner enables flow direction oscillation. The frequency at which the pinch valves 28 switched between open and closed may be set by the user as could the flow rate of the pump 18 (e.g., via the control system 22). Alternative embodiments may pre-program such parameters into the management system 19.

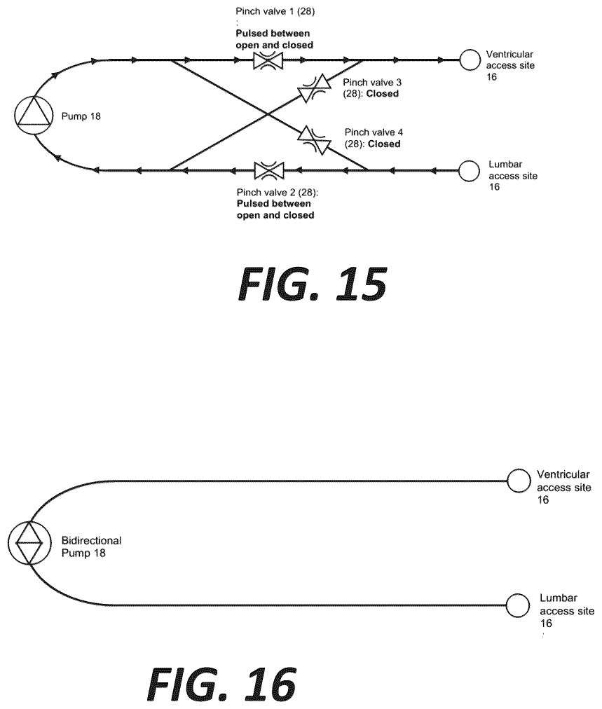

In fact, the same pinch valve 28 configuration (FIG. 15) may be used to create a pulsatile flow pattern. For example, when flowing from lumbar to ventricle, pinch valves 3 and 4 remain closed, while pinch valves 1 and 2 are pulsed (i.e., periodically switched between open and closed) at a frequency set by the user.

The ability to set the frequency at which the pinch valves 28 open and close enables a range of pulsatile effects to be implemented. For example, rather than rapidly switching between open and closed pinch valves 28, the valves 28 can remain closed long enough to build up a set pressure in the fluid line. Shortly after opening the pinch valves 28, a bolus of the drug can be released as a result of the pressure build-up.

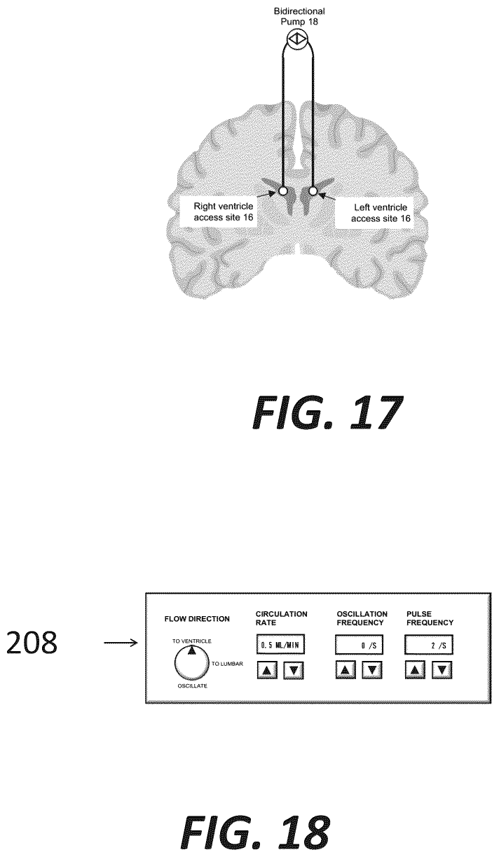

Flow direction oscillation and a pulsatile flow pattern could also be produced using a bidirectional pump 18 instead of using pinch valves 28 (e.g., FIG. 16 and FIG. 17). The pump 18 can be programmed to switch flow directions at a frequency set by the user. While flowing in one direction, the pump 18 can be programmed to pulse by starting and stopping at a frequency also set by the user. Those skilled in the art may use other techniques to provide bidirectional flow.

In addition to those noted above, some embodiments may set the frequency, flow rate, and other parameters as a function of the requirements and structure of the anatomy and devices used in the treatment (e.g., in the CSF circuit 10). Among others, those requirements may include the diameter of the catheters in the CSF circuit 10, physical properties of the drug, the interaction of the drug at the localized region, the properties of the localized region, and other requirements and parameters relevant to the treatment. Those skilled in the art may select appropriate parameters as a function of the requisite properties.

FIG. 18 schematically shows another system interface 20 configured in accordance with illustrative embodiments. Specifically, whether controlling delivery parameters by pinch valve, a bidirectional pump 18, or other means, the delivery profile can be controlled manually with an interface 20, such as the interface 20 shown in FIG. 18, and/or a delivery profile loaded onto the management system 19. As with the other interfaces, this interface 20 may be a fixed control panel, or a graphical user interface on a display device.

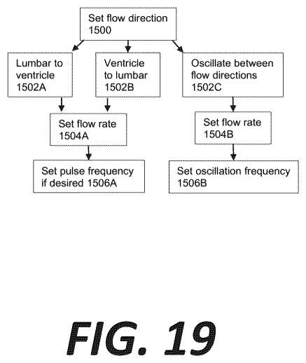

FIG. 19 shows a process of programming drug delivery in accordance with illustrative embodiments. In a manner similar to the other process discussed above, this process is substantially simplified from a longer process that normally would be used to complete the localize drug delivery. Accordingly, this process may have many additional steps that those skilled in the art likely would use. In addition, some of the steps may be performed in a different order than that shown, or at the same time. Those skilled in the art therefore can modify the process as appropriate. Moreover, as noted above and below, many of the materials, devices, and structures noted are but one of a wide variety of different materials and structures that may be used. Those skilled in the art can select the appropriate materials and structures depending upon the application and other constraints. Accordingly, discussion of specific materials, devices, and structures is not intended to limit all embodiments.

The process begins at step 1500, which sets the flow direction. Three options include lumbar to ventricle (1502A), ventricle to lumbar (step 1502B), or oscillating between flow directions (step 1502C). Next, the process sets the flow rate at steps 1504A or 1504B, and sets the frequency of the pulse (step 1506A) or oscillation frequency (step 1506B). Alternative embodiments can be programmed using artificial intelligence algorithms or other program logic.

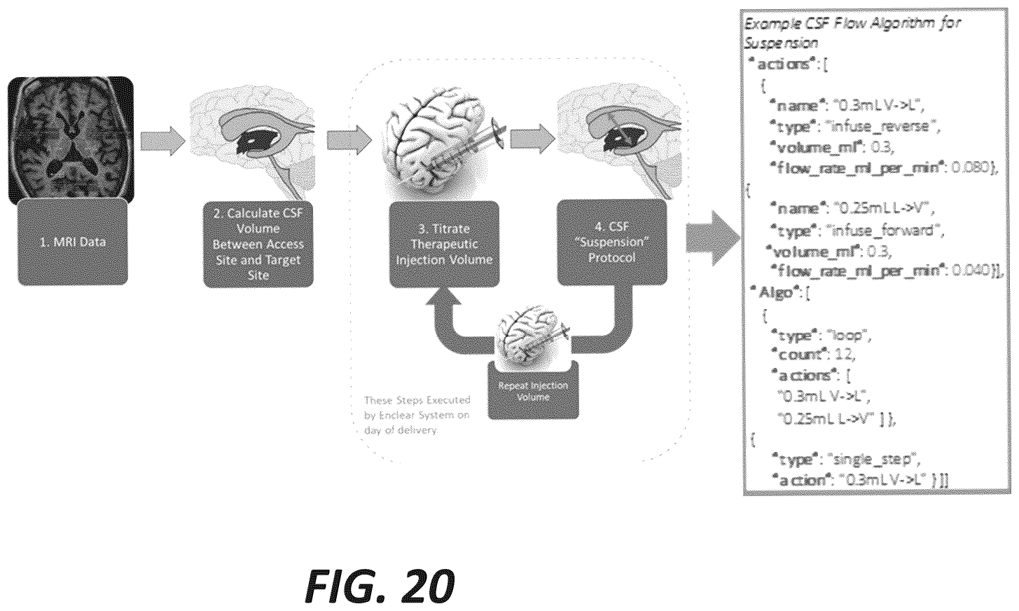

FIG. 20 schematically shows an example of a process in accordance with illustrative embodiments and an exemplary flow algorithm.

As shown, volumetric imaging data (e.g., MRI images) are used to generate the subject's specific cranial CSF volume. Subsequently, the total volume of CSF between the access site and the target site is assessed. Next, if total therapeutic injection volumes exceed this access-to-target volume, injection volumes are broken down into increments matching the access-to-target volume. After this volume is injected, CSF is oscillated to localize therapy over the target. The CSF localization protocol is built from the algorithm that controls the CSF pump, an example of which is displayed to the right side of FIG. 20. After a predetermined oscillation time frame, the next increment of therapeutic injection can be administered. This loop may be repeated until the total desired therapeutic volume has been delivered.

It should be noted that each disease is treated on a personalized basis in each patient. The therapeutic infusion volume is based on a number of factors including: 1) the disease itself, 2) the anatomical aim/location of the therapeutic delivery, and 3) the patient's own anatomy and physiology. Illustrative embodiments provide a system 10 that adjusts to these three variables provides for effective and personalized therapeutic delivery to the CNS.

In the setting of widespread industry failure within CNS drug programs, the ability to deliver compounds to target tissues without other “off target” tissue toxicity has been a limitation solved by illustrative embodiments. For example, illustrative embodiments may enable the therapeutic material to penetrate the basal ganglia, but not penetrate the spinal root nerves, all for a broad patient population.

Patient specific anatomy and physiology comprises of various patient specific factors. To personalize delivery in such a specific manner for a non-homogenous patient population, there are major factors that illustrative embodiments account for including a given patient's specific CNS anatomy, as defined by factors including the shape and volume of their CSF carrying spaces. CSF spaces include the ventricular system, and the subarachnoid space and CSF volumes vary with age, weight, and gender among other factors. For example, a less compliant brain may require a different flow rate or amount of flow time to have a drug reach the same target than in a more compliant brain, even if overall structures are roughly equal.

As an example, if the target structure is near the third ventricle, the system 10 determines the volume of CSF between the where the drug is administered (lateral ventricle), and the target region (the third ventricle). This lateral to third ventricular volume is used to divide a larger bolus for administration. Similarly, after the bolus is divided, the system determines the flow program after CSF passes over the third ventricle, reverses, then forwards and reverses again (oscillates) such that repeat exposures are accomplished and drug levels in the CSF are sustained and the dwell time in the CSF is prolonged. The goal is for drug levels to stay high in the CSF for prolonged periods of time to optimize the equilibrium in the ventricles and interstitial spaces so the drug can reach efficacious concentrations in the targeted brain regions.

Employing live imaging, the controller 22 can develop an algorithm for precision drug delivery. After imaging, a drug, such as methotrexate used for treating brain cancer, can be administered. At necropsy, CNS and peripheral drug tissue concentrations can be quantified to confirm the ability for precision drug delivery and to avoid “off-target” drug delivery. There can be multiple protocols performed under fluoroscopic imaging with the use of Ioxehal. Each animal has MRI images used to measure anatomical properties such as volumes of CNS compartments, together with the results from the fluoroscopy. The contrast agent allows identification of flow characteristics, dwell time, and circulation reversal effects; and it washes out after about 30 minutes, allowing multiple data sets to be collected in a single animal. Ioxehal was administered with an infusion pump (0.125 mL/min with 0.5 mLs volume injected). Illustrative embodiments may provide a variety of circulation protocols, including:
1. Circulation from ventricle to ventricle with use of controlled flow reversal and oscillation as determined by imaging;
2. Circulation from ventricle to lumbar spine with use of controlled flow reversal and oscillation as determined by imaging; and/or
3. Circulation from ventricle to lumbar spine with controlled flow unidirectional.

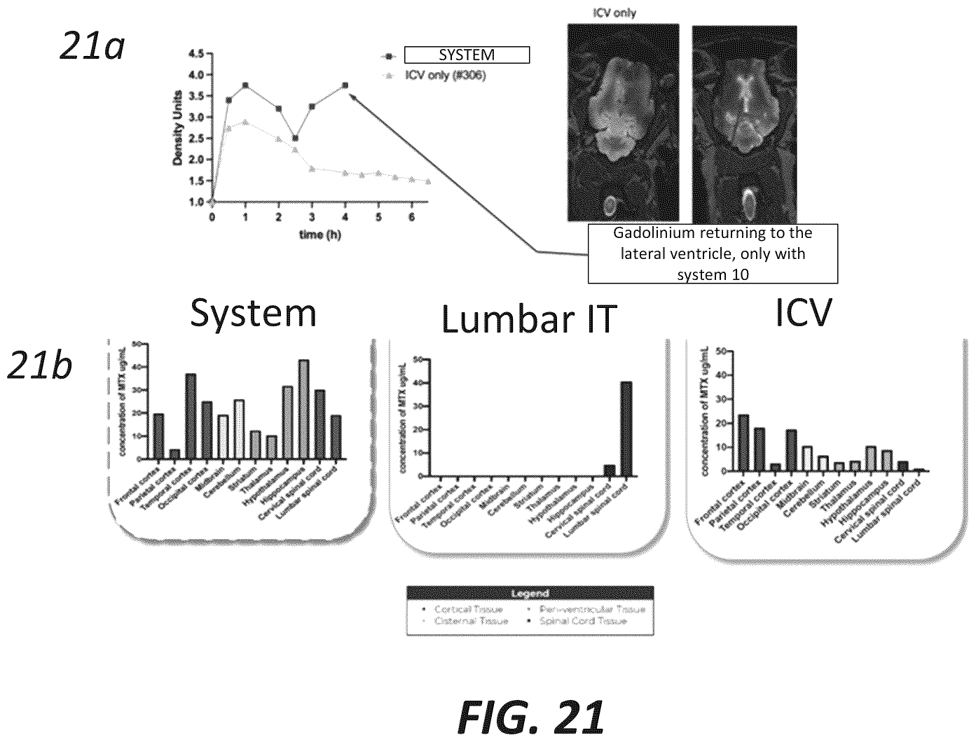

FIG. 21 shows exemplary data using CSF flow oscillation in the cranium. In animal studies conducted thus far, the system 10 generates flow rate ranging from 20 to 400 μL/min, which is expected to be in the range of human clinical setting. The experimental data demonstrates broad drug distribution in the CNS. The drug methotrexate was co-injected with gadolinium into the lateral ventricle and MRI and drug was measured in CSF and tissues. (21a) shows both the quantitation and image of how the system 10 generates a constant fluidic circuit which leads to two peaks of intensity by recirculating the tracer and methotrexate from the lateral ventricle, through the spine, through the device, and back into the lateral ventricle. Notably, the intensity value returns, as compared to the ICV only injection, which slowly diffuses out of the CSF. (21b) depicts the tissue distribution of the methotrexate. The improved CSF residency time translated to improved methotrexate distribution across the CNS when compared to standard of care LIT and ICV controls. This provided initial evidence that the system's 10 flow mechanism maintains concentrations in the CSF and that this improves parenchymal uptake. Data is from one representative sheep.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

In some implementations, the controller 22 includes one or more processors (or one or more processor cores) that each are configured to perform a series of instructions that result in manipulated data and/or control the operation of the other components of the controller 22. In some implementations, when executing a specific process (e.g., determining the personalized therapeutic infusion volume), the processor can be configured to make specific logic-based determinations based on input data received, and be further configured to provide one or more outputs that can be used to control or otherwise inform subsequent processing to be carried out by the processor and/or other processors or circuitry with which processor is communicatively coupled. Thus, the processor reacts to specific input stimulus in a specific way and generates a corresponding output based on that input stimulus. In some example cases, the processor can proceed through a sequence of logical transitions in which various internal register states and/or other bit cell states internal or external to the processor may be set to logic high or logic low. As referred to herein, the processor can be configured to execute a function where software is stored in a data store coupled to the processor, the software being configured to cause the processor to proceed through a sequence of various logic decisions that result in the function being executed. The various components that are described herein as being executable by the processor can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

As used in this specification and the claims, the singular forms “a,” “an,” and “the” refer to plural referents unless the context clearly dictates otherwise. For example, reference to “the drug” in the singular includes a plurality of drugs, and reference to “the pump” in the singular includes one or more pumps and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A CSF management method for use with a patient having a body with CSF having a natural flow rate, the method comprising:
determining a target volume of a CSF-containing target compartment of a patient from a volumetric image;
determining a personalized therapeutic infusion volume of a therapeutic material as a function of the target volume and a total therapeutic infusion volume;
forming a CSF circuit to control flow of CSF in the body;
adding the personalized therapeutic infusion volume to the CSF via the CSF circuit at a first time;
directing the therapeutic material, via the CSF, toward the target compartment of the patient; and
adjusting the flow of the CSF in the CSF circuit to localize the CSF at the target compartment of the patient.

2. The method of claim 1, wherein adjusting the flow of the CSF comprises oscillating the flow of the CSF.

3. The method of claim 1, wherein the personalized therapeutic infusion volume is a fraction of the total therapeutic infusion volume.

4. The method of claim 1, further comprising:
determining a concentration of the therapeutic material in the CSF from the personalized therapeutic infusion volume, and when the concentration drops below a given threshold, adding a second personalized therapeutic infusion volume to the CSF via the CSF circuit at a second time subsequent to the first time.

5. The method of claim 1, wherein the volume of the personalized therapeutic infusion volume is less than or the same as the target volume.

6. The method of claim 1, wherein the target volume comprises a delivery volume.

7. The method of claim 6, wherein the target volume includes a peripheral volume.

8. A CSF management system comprising:
a catheter configured to fluidly couple with a CSF-containing space of a patient and a drug infusion pump;
a bidirectional drug infusion pump configured to provide a drug to the patient via the catheter;
a controller configured to:
determine a personalized therapeutic infusion volume of therapeutic material to provide to a patient as a function of a received target volume of a CSF-containing target space of the patient,
control the bidirectional drug infusion pump to provide the personalized therapeutic infusion volume to the patient via the catheter, and
adjust flow of the CSF in the patient to localize the CSF at the target space of the patient.

9. The system of claim 8, further comprising a target volume calculator configured to determine the target volume from a volumetric image.

10. The system of claim 8, further comprising a therapeutic material position analyzer configured to determine a position of the therapeutic material, wherein the position of the therapeutic material is used as feedback to control the bidirectional infusion pump to adjust the flow of the CSF to maintain a desired position of the therapeutic material.

11. The system of claim 8, further comprising a drug concentration analyzer configured to determine a concentration of the therapeutic material in the CSF, wherein the drug concentration analyzer is configured to use the concentration as feedback to control the delivery of a second personalized therapeutic infusion volume of therapeutic material.

12. The system of claim 8, further comprising a sensor configured to determine a pressure in the CSF space, wherein the pressure is used to determine the compliance of the brain and adjust the target volume.

13. The system of claim 8, wherein the bidirectional pump is configured to adjust the flow of the by oscillating the flow of the CSF.

14. A computer program product for use on a computer system for managing CSF flow within a human patient having CSF, the computer program product comprising a tangible, non-transient computer usable medium having computer readable program code thereon, the computer readable program code comprising:
program code for determining a target volume of a CSF-containing target compartment of a patient from a volumetric image;
program code for determining a personalized therapeutic infusion volume of a therapeutic material as a function of the target anatomical volume and a total therapeutic infusion volume;
program code for forming a CSF circuit to control flow of CSF in the body;
program code for controlling a pump to cause the personalized therapeutic infusion volume to flow to the CSF via the CSF circuit at a first time;
program code for controlling a pump to direct the therapeutic material, via the CSF, toward the target compartment of the patient; and
program code for controlling a pump to adjust the flow of the CSF in the CSF circuit to localize the CSF at the target compartment of the patient.

15. The computer program product of claim 14, further comprising:
program code for determining a concentration of the therapeutic material in the CSF from the personalized therapeutic infusion volume,
program code for adding a second personalized therapeutic infusion volume to the CSF via the CSF circuit when the concentration drops below a given threshold.

16. The computer program product of claim 14, wherein the personalized therapeutic infusion volume is less than or the same as the target volume.

17. The computer program product of claim 14, further comprising program code for determining the target volume as a function of brain.

18. The computer program product of claim 14, further comprising program code for obtaining the volumetric image is from an MRI.

19. The computer program product of claim 14, wherein the CSF circuit accesses one or more CSF-containing compartments with patient anatomy, including one or more of the lateral ventricles, the lumbar thecal sac, the third ventricle, the fourth ventricle, and the cisterna magna.

20. The computer program product of claim 14, further comprising program code for oscillating the flow of CSF within the CSF circuit for a prescribed time and at a prescribed frequency.
